(19) United States
(12) Patent Application Publication
(10) Pub. No.: US 2021/0401076 A1
(43) Pub. Date: Dec. 30, 2021

(54) ARM PROTECTOR KITS AND METHODS OF MAKING AND USING THE SAME

(71) Applicant: GRI-ALLESET, INC., Flowery Branch, GA (US)

(72) Inventors: Brian E. Jenkins, Cumming, GA (US); Cayd Ayres, Knoxville, TN (US); Simon Alesbury, Surbiton (GB); Parker Hoda, Atlanta, GA (US)

(21) Appl. No.: 17/355,539

(22) Filed: Jun. 23, 2021

Related U.S. Application Data
(60) Provisional application No. 63/043,219, filed on Jun. 24, 2020.

(51) Int. Cl. A41D 13/08

(52) U.S. Cl. CPC A41D 13/08

(57) ABSTRACT

Arm protector kits are disclosed. Methods of making and using arm protector kits are also disclosed.

### RELATED APPLICATIONS

This patent application claims the benefit of priority to U.S. Provisional Patent Application Ser. No. 63/043,219 filed on Jun. 24, 2020 and entitled “ARM PROTECTOR KITS AND METHODS OF MAKING AND USING THE SAME,” the subject matter of which is hereby incorporated by reference in its entirety.

### TECHNICAL FIELD

The present invention relates to arm protectors suitable for use during an operating procedure.

### BACKGROUND

There is a need in the art for improved arm protectors suitable for use during an operating procedure.

### SUMMARY

The present invention addresses a need in the arm protector art by providing an improved arm protector kit suitable for use during an operating procedure. Accordingly, the present invention is directed to an arm protector kit.

In some embodiments, the arm protector kit of the present invention comprises (a) a composite pad sized to extend around and along an arm of a patient, said composite pad having a pad thickness, a pad length and a pad width, and comprising a foam layer and at least one loop material layer along a major outer surface of the foam layer; (b) a wrist support component that is attachable to, repositionable on, and removable from a major outer surface of said composite pad, said wrist support component comprising a foam arch; and (c) at least three tabs, each tab (i) being attachable to, repositionable on, and removable from a major outer surface of said composite pad, and (ii) comprising a strip of hook material having a strip length and a strip width, wherein the arm protector kit forms a protective arm wrapping with a closed end that encapsulates and protects fingers of the patient when assembled on the arm of the patient.

The present invention is further directed to methods of making the disclosed arm protector kits. In one exemplary embodiment, the method of making the disclosed arm protector kit comprises combining the herein-described composite pad, the herein-described wrist support component, and the herein-described at least three tabs with one another. Methods of making the disclosed arm protector kits may comprise one or more additional steps such as attaching the wrist support component and the at least three tabs to the composite pad; a composite pad forming step; a wrist support component forming step; a tab forming step; folding the composite pad along a length direction D_{L} of the composite pad; and packaging the composite pad, the wrist support component and the at least three tabs within one or more pieces of packaging material.

The present invention is further directed to methods of using the disclosed arm protector kits. In one exemplary embodiment, the method of using the disclosed arm protector kit of the present invention comprises forming a protective arm wrapping with a closed end that encapsulates and protects fingers of a patient, for example, as during a medical procedure. Methods of using the disclosed arm protector kits may further comprise one or more of the following steps: positioning the wrist support component so as to attach to a central portion of a first major outer surface of the composite pad proximate a distal end of the composite pad; positioning the composite pad along and under the patient's arm so that the fingers of the patient extend along an upper surface of the foam arch at a distal end of the foam arch; folding an outer edge third portion of the composite pad along a length direction D_{L} of the composite pad so that the outer edge third portion of the composite pad is positioned over the central portion of the composite pad; folding an opposite outer edge third portion of the composite pad along the length direction D_{L} so that the opposite outer edge third portion of the composite pad is positioned over the central portion of the composite pad and a lower surface portion of the outer edge third portion of the composite pad; attaching two tabs of the at least three tabs to the composite pad so as to secure (i) the opposite outer edge third portion of the composite pad and (ii) the outer edge third portion of the composite pad to one another, the two tabs extending in a width direction D_{W} of the composite pad; and attaching one tab of the at least three tabs to the composite pad so as to secure (i) a lower surface of the central portion of the composite pad and (ii) a lower surface of the opposite outer third portion of the composite pad to one another, the one tab extending in a length direction D_{L} of the composite pad and forming the closed end of the protective arm wrapping.

These and other features and advantages of the present invention will become apparent after a review of the following detailed description of the disclosed embodiments and the appended claims.

### BRIEF DESCRIPTION OF THE DRAWINGS

The present invention is further described with reference to the appended figures, wherein:

FIG. 1 depicts an exemplary arm protector kit of the present invention;

FIG. 2 depicts a close-up view of an exemplary wrist support component suitable for use in the arm protector kits of the present invention;

FIG. 3 depicts a close-up view of exemplary tabs suitable for use in the arm protector kits of the present invention;

FIGS. 4A-4E depict an exemplary procedure for forming a protective arm wrapping over a patient's arm using an exemplary arm protector kit of the present invention;

FIG. 5 depicts an exemplary cross-sectional view of an exemplary composite pad suitable for use in the arm protector kits of the present invention;

FIG. 6 depicts an exemplary side view of an exemplary wrist support component suitable for use in the arm protector kits of the present invention;

FIGS. 7A-7E depict an exemplary procedure for folding an exemplary composite pad so as to nest an exemplary wrist support component of the arm protector kits of the present invention;

FIG. 8 depicts various packaging materials that may be used to package the arm protector kits of the present invention;

FIGS. 9A-9E depict another exemplary arm protector kit of the present invention, wherein the exemplary arm protector kit is for a right-handed patient, and comprises a numerical system for assisting with folding of the exemplary arm protector kit; and

FIGS. 10A-10E depict another exemplary procedure for folding exemplary composite pads so as to nest left and right hands of a user along exemplary wrist support components and within exemplary arm protector kits of the present invention.

### DETAILED DESCRIPTION OF THE INVENTION

The present invention is directed to arm protector kits. The present invention is further directed to methods of making arm protector kits. The present invention is even further directed to methods of using arm protector kits to protect a patient's arm during a surgical procedure.

The arm protector kits of the present invention provide one or more of the following advantages over known arm protectors:

(1) the arm protector kits of the present invention are universal in design, namely, all components can be moved to allow each arm protector to be used for either arm;

(2) the arm protector kits of the present invention have a thickness of foam between the patient's arm and the patient's body so as to not push the patient's arm too far from the surgical table;

(3) the arm protector kits of the present invention have a thickness so as not impinge on the access to the low lateral trocars in robotic and laparoscopic cases (i.e., too much material makes it difficult to access the upper abdomen if placed incorrectly);

(4) the arm protector kits of the present invention provides a unique wrist/hand protector that can be easily repositioned by using our “lift tabs” attached to each end of the wrist/hand protector, the user can easily pull and move the wrist/hand protector without destroying/tearing the composite pad materials;

(5) the arm protector kits of the present invention has a universal length;

(6) the arm protector kits of the present invention have a thickness of material that can be easily cut and/or folded to reduce length for individual patients;

(7) the arm protector kits of the present invention comprise hook & loop tabs that are not sewn/integrated into the wrap composite pad material so that the tabs are easily repositionable, allowing for a patient-specific, customized fit for each patient and procedure;

(8) the arm protector kits of the present invention protect the patient's fingers from possible crushing injury and/or burn given that the user can fold back the available composite pad material at the distal end of the arm protector to close the end of the arm protector, and use one of our hook and loop tabs to secure this folded piece of material, and by doing this, preventing the patient's fingers from protruded from the end of the arm protector;

(9) the arm protector kits of the present invention offer the use a quick set up time, and also reduces the number of people/time required to wrap/protect the patient's arms;

(10) the arm protector kits of the present invention provide ease of access to the patient's arm in case of emergency, as well as easy access to any lines connected to the patient's arm(s);

(11) the arm protector kits of the present invention are made of soft material to accommodate the “fragile/delicate” skin of some patients (elderly, etc. . . . );

(12) the arm protector kits of the present invention have the ability to accommodate a wide range of arm sizes (i.e., arm diameter and arm length); and

(13) the arm protector kits of the present invention comprise a medical device that is specifically designed for arm protection, particularly during a medical procedure.

The arm protector kits of the present invention may comprise a number of components.

A description of individual components and combinations of individual components is provided below.

### I. Arm Protector Kit Components

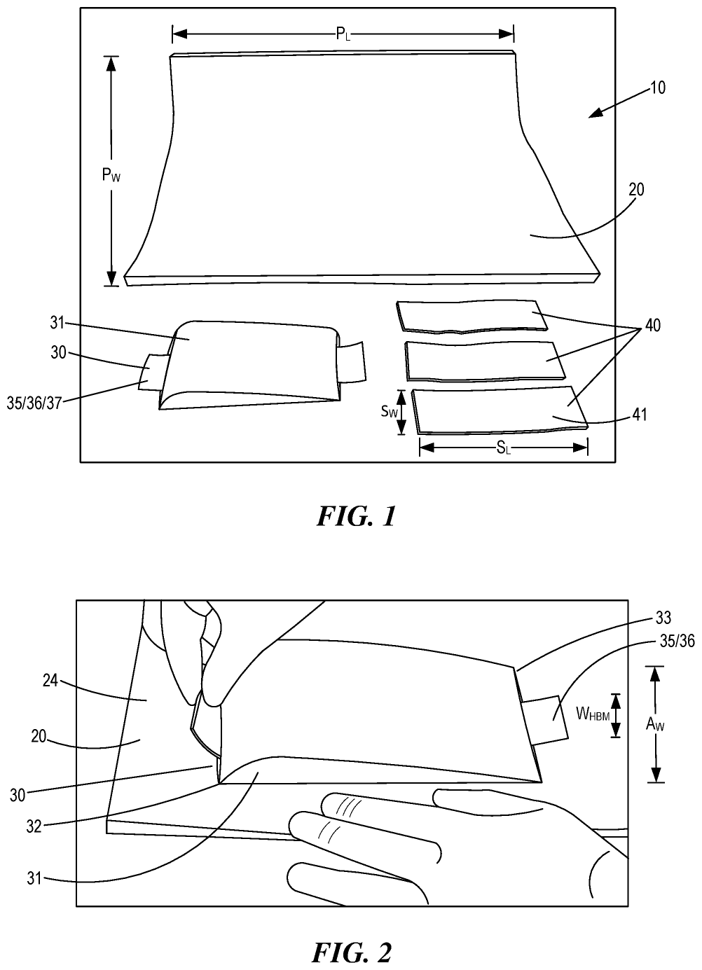

FIG. 1 depicts an exemplary arm protector kit 10 of the present invention. As shown in FIG. 1, arm protector kits of the present invention may comprise one or more of the following components.

A. Composite Pad

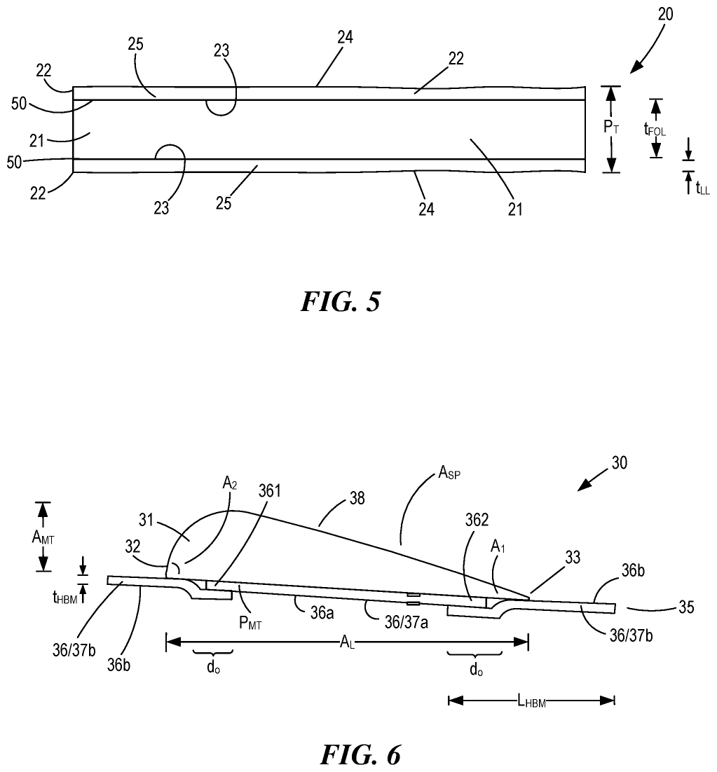

Arm protector kits of the present invention, such as exemplary arm protector kit 10 shown in FIG. 1, comprise a composite pad 20. Composite pad 20 comprises a foam layer 21 and one or more optional additional layers. In some embodiments, composite pad 20 comprises a foam layer 21 in combination with at least one loop material layer 22 bonded to and extending along a major outer surface 23 of the foam layer 21. In other embodiments, composite pad 20 comprises a foam layer 21 in combination with two loop material layers 22 bonded to and extending along opposite major outer surfaces 23 of the foam layer 21. See, for example, FIG. 5.

B. Wrist Support Component

The arm protector kits of the present invention, such as exemplary arm protector kit 10 shown in FIG. 1, further comprise a wrist support component 30. Wrist support component 30 comprising a foam arch 31 as shown in FIGS. 1 and 6. Typically, the wrist support component 30 further comprises a hook backing layer 35, wherein the hook backing layer 35 extends along a lower surface 34 of the foam arch 31. See again, FIG. 6. The hook backing layer 35 may comprise one or more separate pieces 36 of hook backing material 37. In some embodiments, the hook backing layer 35 comprises three separate pieces 36 of backing material 37 with at least one piece 36 of backing material 37 comprises hook material 37a. See, for example, exemplary hook backing layer 35 comprising (a) centrally-positioned piece 36 of backing material 37 comprising hook material 37a, and (b) opposite grasping pieces 36b comprising non-hook material 37b (e.g., a material other than hook material 37a such as a polypropylene webbing) attached to and overlapping end portions 361/362 of centrally-positioned piece 36 in FIG. 6.

C. Tabs

The arm protector kits of the present invention, such as exemplary arm protector kit 10 shown in FIG. 1, further comprise at least three tabs 40. Each tab 40 (i) is attachable to, repositionable on, and removable from a major outer surface 24 of the composite pad 20, and (ii) comprises a strip of hook material 41 having a strip length S_{L} and a strip width S_{W}.

D. Other Optional Components

The arm protector kits of the present invention, such as exemplary arm protector kit 10 shown in FIG. 1, may further comprise one or more of the following optional kit components: a second composite pad 20, a second wrist support component 30, additional tabs 40 (e.g., from 6 to 10 total tabs 40), a patient pad (i.e., for positioning under the patient when positioned on an operating table), a lift sheet, two or more body straps, or any combination thereof.

### II. Methods of Making Arm Protector Kits

The present invention is further directed to methods of making the disclosed arm protector kits. In one exemplary embodiment, the method of making the disclosed arm protector kit comprises combining the composite pad 20, the wrist support component 30, and the at least three tabs 40 with one another. The step of combining may simply comprise attaching the wrist support component 30 and the at least three tabs 40 to the composite pad 20.

The methods of making the disclosed arm protector kits may further comprise one or more additional steps such as a composite pad forming step; a wrist support component forming step; a tab forming step, or any combination thereof.

### III. Methods of Using Arm Protector Kits

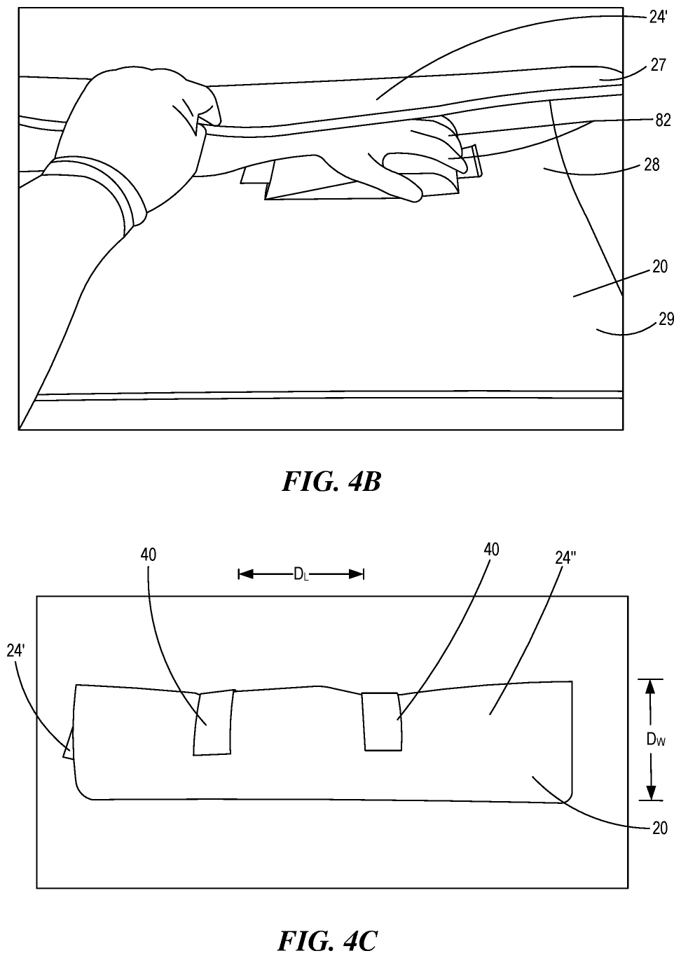

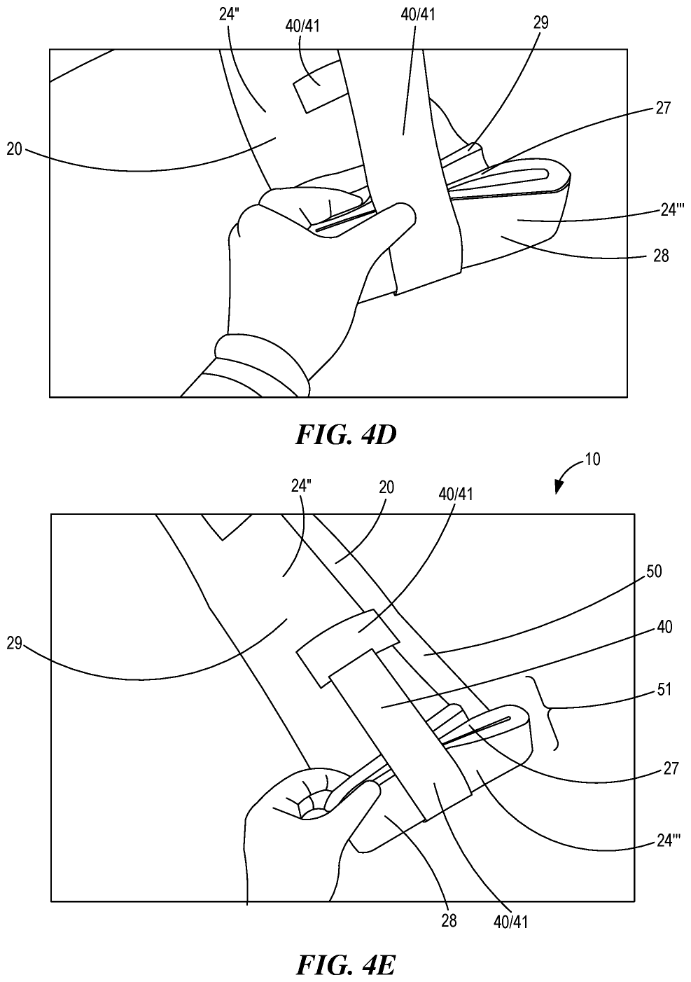

The present invention is further directed to methods of using the disclosed arm protector kits. In one exemplary embodiment, the method of using the disclosed arm protector kit comprises forming a protective arm wrapping 50 with a closed end 51 that encapsulates and protects fingers 82 of the patient 80. See, for example, the exemplary method steps shown in FIGS. 4A-4E, wherein exemplary protective arm wrapping 50 with closed end 51 is formed using wrist support component 30, composite pad 20 and three tabs 40 with two tabs extending in a width direction D_{W} of composite pad 20 and one tab 40 extending in a length direction D_{L} of composite pad 20. As shown in FIG. 4E, exemplary protective arm wrapping 50 with closed end 51 encapsulates and protects fingers 82 of patient 80.

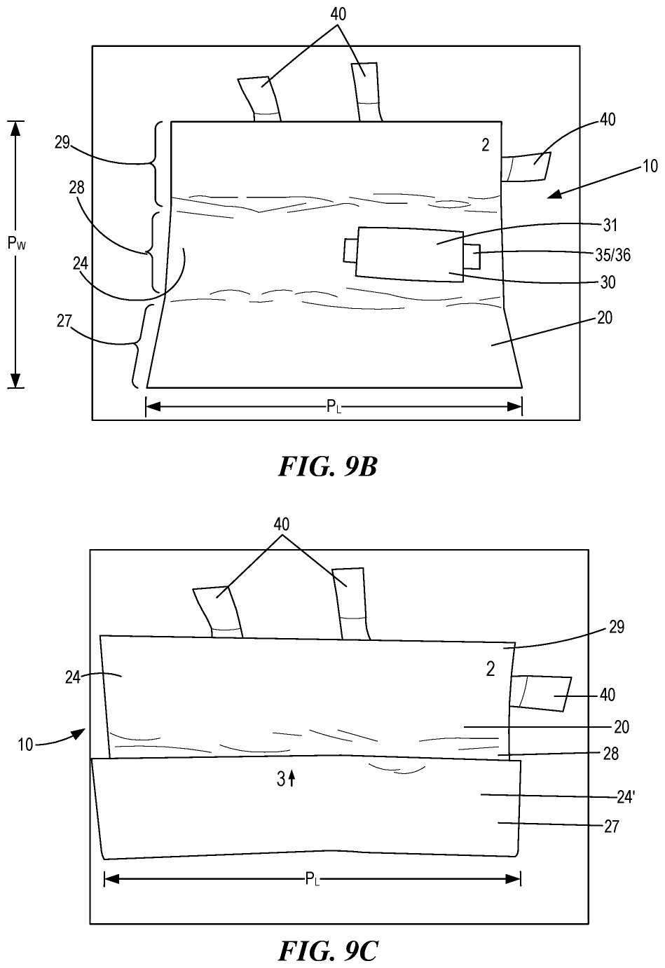

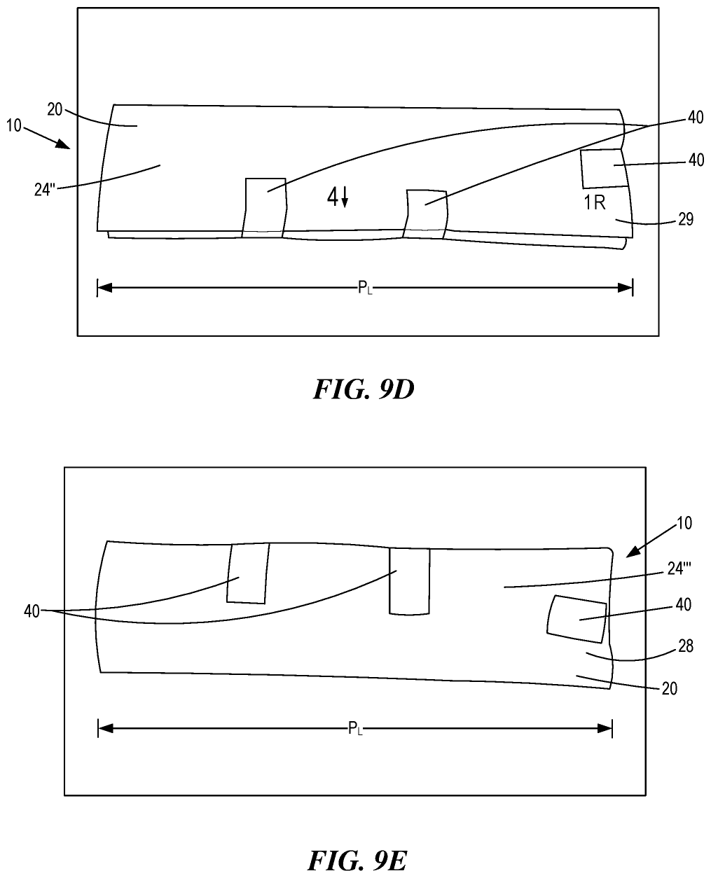

FIGS. 9A-9E provide another exemplary arm protector kit of the present invention, wherein the arm protector kit further comprises a numbering system to assist a user with folding the arm protector kit. As shown in FIG. 9A, exemplary arm protector kit 10 comprises the following lettering/numbering: (a) “1R” along lower surface 24″ of the upper, opposite outer third edge portion 29 of composite pad 20, indicating that this particular arm protector kit 10 is suitable fir use with a right-handed user; (b) “3↑” along lower surface 24′ of the upper, opposite outer third edge portion 27 of composite pad 20; and (c) “4↓” along lower surface 24″ of the upper, opposite outer third edge portion 29 of composite pad 20. As shown in FIG. 9B, exemplary arm protector kit 10 further comprises (d) “2” along major outer surface 24 in an upper, right-hand corner of the upper, opposite outer third edge portion 29 of composite pad 20.

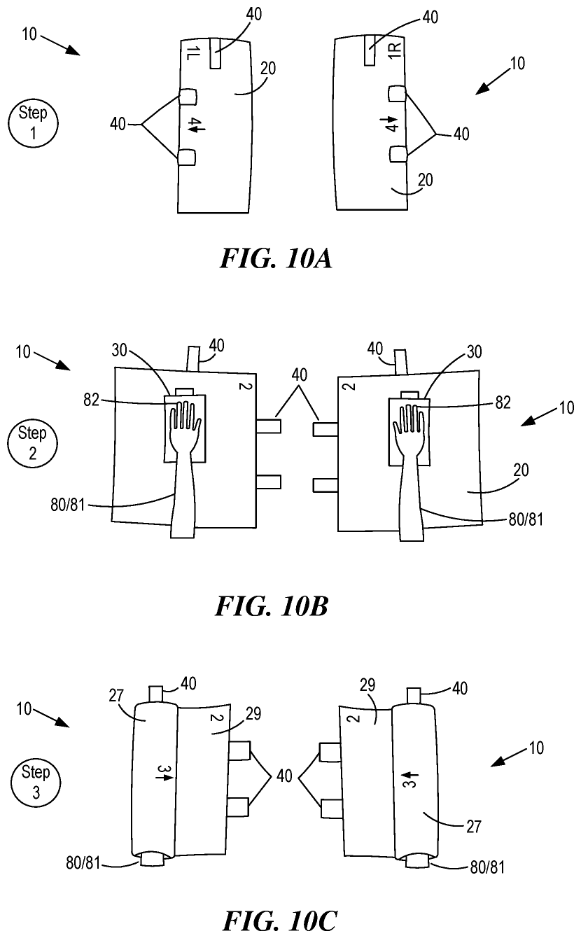

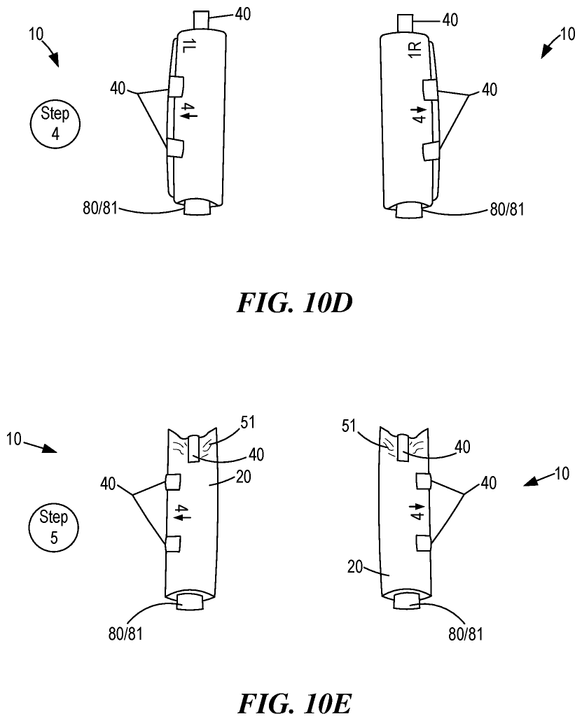

FIGS. 10A-10E depict method steps for using arm protector kits 10 of the present invention for wrapping both a left-handed (i.e., identified as “1L”) and a right-handed (i.e., identified as “1R”) wrist of users. As shown in FIG. 10A, in Step 1, a user opens the arm protector kit 10 by lifting the two tabs extending in a width direction D_{W} of composite pad 20 and the one tab 40 extending in a length direction D_{L} of composite pad 20. Each tab 40 can be carefully unhooked from one outer surface of the composite pad 20 without completely removing a given tab 40 from composite pad 20.

As shown in FIG. 10B, in Step 2, with the composite pad 20 spread open, a given composite pad 20 (i.e., identified as composite pad “1L” for the left-handed wrist, and identified as composite pad “1R” for the right-handed wrist) is positioned under a patient's arm so that the patient's hand rests along an arch upper surface profile A_{SP} of the wrist support component 30 with the palm of the patient facing downwards. The wrist is desirably placed so that there is enough distance (e.g., a minimum of about 3 inches) between the fingertips of the patient and the edge of the composite pad 20 (so that the end of the composite pad 20 can be pulled downward back towards the top face of the composite pad 20 as shown in Step 5 below).

As shown in FIG. 10C, in Step 3, a user locates the “3↑” symbol, and pulls the edge adjacent the “3↑” symbol over the patient's arm and holds the edge in place.

As shown in FIG. 10D, in Step 4, a user locates the “4↓” symbol, and pulls the edge adjacent the “4↓” symbol over the “3↑” symbol and the patient's arm, and secures the edge in place with the two tabs 40 extending along the length P_{L} of composite pad 20.

As shown in FIG. 10E, in Step 5, a user folds top end of the composite pad 20 back towards the top face of the composite pad 20, and secures the folded portion of the composite pad 20 onto an inner portion of the composite pad 20 with the single tab 40 extending along the width P_{W} of composite pad 20.

Additional embodiments of the present invention are provided in detail below.

### Other Embodiments

Arm Protector Kits:

1. An arm protector kit 10 comprising: a composite pad 20 sized to extend around and along an arm 81 of a patient 80, said composite pad 20 having a pad thickness P_{T}, a pad length P_{L} and a pad width P_{W}, and comprising a foam layer 21 and at least one loop material layer 22 along a major outer surface 23 of the foam layer 21; a wrist support component 30 that is attachable to, repositionable on, and removable from a major outer surface 24 of said composite pad 20, said wrist support component 30 comprising a foam arch 31; and at least three tabs 40, each tab 40 (i) being attachable to, repositionable on, and removable from a major outer surface 24 of said composite pad 20, and (ii) comprising a strip of hook material 41 having a strip length S_{L} and a strip width S_{W}, wherein the arm protector kit 10 forms a protective arm wrapping 50 with a closed end 51 that encapsulates and protects fingers 82 of the patient 80 when assembled on the arm 81 of the patient 80. See, for example, FIGS. 1 and 4E.

2. The arm protector kit 10 of embodiment 1, wherein said at least one loop material layer 22 comprises two loop material layers 22 with one loop material layer 22 extending along opposite major outer surfaces 23 of the foam layer 21. See, for example, FIG. 5.

3. The arm protector kit 10 of embodiment 1 or 2, wherein said foam layer 21 comprises a single foam layer 21. See, for example, FIG. 5.

4. The arm protector kit 10 of any one of embodiments 1 to 3, wherein the foam layer 21 has a layer density of from about 0.5 to about 5.0 lb density.

5. The arm protector kit 10 of any one of embodiments 1 to 4, wherein the foam layer 21 has a layer density of from about 1.0 to about 2.0 lb density. In some embodiments, the foam layer 21 has a layer density of about 1.4 lb.

6. The arm protector kit 10 of any one of embodiments 1 to 5, wherein the foam layer 21 has an Impression Load Deflection (ILD) value of from about 36 to about 50.

7. The arm protector kit 10 of any one of embodiments 1 to 6, wherein the foam layer 21 has an Impression Load Deflection (ILD) value of from about 40 to about 50.

8. The arm protector kit 10 of any one of embodiments 1 to 7, wherein the foam layer 21 has a layer thickness t_{FOL} of from about 0.5 centimeter (cm) to about 2.0 cm. See, for example, FIG. 5.

9. The arm protector kit 10 of any one of embodiments 1 to 8, wherein the foam layer 21 has a layer thickness t_{FOL} of about 1.0 cm.

10. The arm protector kit 10 of any one of embodiments 1 to 9, wherein the foam layer 21 comprises a polyurethane foam, a polyester foam, a polyester polyurethane foam, or a combination thereof. In some embodiments, the foam layer 21 comprises a polyester polyurethane foam.

11. The arm protector kit 10 of any one of embodiments 1 to 10, wherein the foam layer 21 comprises a polyurethane non-viscoelastic foam, a polyester non-viscoelastic foam, a polyester polyurethane non-viscoelastic foam, or a combination thereof.

12. The arm protector kit 10 of any one of embodiments 1 to 11, further comprising an adhesive layer 50 between the foam layer 21 and each of said at least one loop material layer 22.

13. The arm protector kit 10 of any one of embodiments 1 to 12, wherein each of said at least one loop material layer 22 comprises loop material 25 comprising of a layer of warp unbroken loop (UBL) knit fabric. In some embodiments, the warp unbroken loop (UBL) knit fabric comprises a 2.75 ounce per square yard (osy) 100% nylon warp unbroken loop (UBL) knit fabric. See, for example, FIG. 5.

14. The arm protector kit 10 of any one of embodiments 1 to 13, wherein each of said at least one loop material layer 22 has a loop layer thickness t_{LL} of about 0.2 cm.

15. The arm protector kit 10 of any one of embodiments 1 to 14, wherein said composite pad 20 has a pad thickness P_{T} of from about 0.5 cm to about 2.0 cm, a pad length P_{L} of from about 50.0 cm to about 90.0 cm, and a pad width P_{W} of from about 40.0 cm to about 70.0 cm.

16. The arm protector kit 10 of any one of embodiments 1 to 15, wherein said composite pad 20 has a pad thickness P_{T} of about 1.0 cm, a pad length P_{L} of about 67.0 cm, and a pad width P_{W} of about 55.0 cm.

17. The arm protector kit 10 of any one of embodiments 1 to 16, wherein said wrist support component 30 is attachable to, repositionable on, and removable from a major outer surface 24 of said composite pad 20 without causing damage to the major outer surface 24 of said composite pad 20.

18. The arm protector kit 10 of any one of embodiments 1 to 17, wherein said foam arch 31 has an arch maximum thickness A_{MT} of from about 2.5 cm to about 6.0 cm, an arch length A_{L} of from about 15.0 cm to about 28.0 cm, and an arch width A_{W} of from about 6.0 cm to about 15.0 cm. See, for example, FIGS. 2 and 6.

19. The arm protector kit 10 of any one of embodiments 1 to 18, wherein said foam arch 31 has an arch maximum thickness A_{MT} of about 4.0 cm, an arch length A_{L} of about 21.0 cm, and an arch width A_{W} of about 10.0 cm.

20. The arm protector kit 10 of embodiment 18 or 19, wherein said arch maximum thickness A_{MT} is closer to a distal end 32 of said foam arch 31 than a proximal end 33 of said foam arch 31.

21. The arm protector kit 10 of any one of embodiments 18 to 20, wherein said arch maximum thickness A_{MT} is from about 3.0 to about 4.0 cm from a distal end 32 of said foam arch 31.

22. The arm protector kit 10 of any one of embodiments 18 to 21, wherein said foam arch 31 has an arch upper surface profile A_{SP} that gradually increases in height (i.e., thickness) from a proximal end 33 of said foam arch 31 to a point P_{MT} along the arch length A_{L} of said foam arch 31 that contains the arch maximum thickness A_{MT} of said foam arch 31, and then curves downward towards a distal end 32 of said foam arch 31. See, for example, FIGS. 2 and 6.

23. The arm protector kit 10 of any one of embodiments 18 to 22, wherein said foam arch 31 has an arch upper surface profile A_{SP} that forms (i) a first angle A₁ with a lower surface 34 of said foam arch 31 at a proximal end 33 of said foam arch 31, and (ii) a second angle A₂ with the lower surface 34 of said foam arch 31 at a distal end 32 of said foam arch 31, and wherein the first angle A₁ is less than about 15° and the second angle A₂ is from about 60° to about 90°. See, for example, FIG. 6.

24. The arm protector kit 10 of any one of embodiments 1 to 23, wherein said foam arch 31 comprises a polyurethane non-viscoelastic foam, a polyester non-viscoelastic foam, a polyester polyurethane non-viscoelastic foam, or a combination thereof.

25. The arm protector kit 10 of any one of embodiments 1 to 24, wherein said wrist support component 30 further comprises a hook backing layer 35, said hook backing layer 35 extending along a lower surface 34 of said foam arch 31.

26. The arm protector kit 10 of embodiment 25, wherein said hook backing layer 35 comprises one or more separate pieces 36 of backing material 37, wherein at least one piece 36 of backing material 37 comprises a hook material 37a. The one or more separate pieces 36 of backing material 37 may be used separately (e.g., each piece 36 of backing material 37 is individually attached to lower surface 34 of foam arch 31) or in combination with one another (e.g., one piece 36 of backing material 37 is attached to lower surface 34 of foam arch 31 and other piece(s) 36 of backing material 37 is/are attached to the one piece 36 of backing material 37 that is attached to lower surface 34 of foam arch 31).

27. The arm protector kit 10 of embodiment 25 or 26, wherein said hook backing layer 35 comprises two or more separate pieces 36 of backing material 37.

28. The arm protector kit 10 of any one of embodiments 25 to 27, wherein said hook backing layer 35 comprises three separate pieces 36 of backing material 37. See, for example, FIG. 6.

29. The arm protector kit 10 of any one of embodiments 26 to 28, wherein at least one piece 36 of backing material 37 comprises a hook material, for example, a 2″ wide White Hook, 100% nylon hook material.

30. The arm protector kit 10 of any one of embodiments 26 to 29, wherein at least one piece 36 of backing material 37 (e.g., hook backing 37a) has a backing material thickness t_{HBM} of less than about 0.3 cm, a backing material length L_{HBM} of from about 6.0 cm to about 18.0 cm, and a backing material width W_{HBM} of from about 4.0 cm to about 8.0 cm. See, for example, FIGS. 2 and 6.

31. The arm protector kit 10 of any one of embodiments 26 to 30, wherein at least one piece 36 of backing material 37 (e.g., each of the two grasping pieces 36b) has a backing material length L_{HBM} of (i) from about 6.0 cm to about 8.0 cm or (ii) from about 16 cm to about 18 cm, and a backing material width W_{HBM} of about 5.0 cm.

32. The arm protector kit 10 of any one of embodiments 25 to 31, wherein said hook backing layer 35 comprises (1) a centrally positioned first piece 36a of backing material 37 comprising the hook material 37a with (i) a backing material length L_{HBM} of about 17.0 cm and (ii) a backing material width W_{HBM} of about 5.0 cm, and (2) two grasping pieces 36b of backing material 37, each independently comprising, for example, a polypropylene fiber webbing, having (i) a backing material length L_{HBM} of about 7.0 cm and (ii) a backing material width W_{HBM} of about 5.0 cm, wherein each of the two grasping pieces 36b of backing material 37 extend beyond a lower surface 34 of the foam arch 31 at opposite distal and proximal ends 32/33 of said foam arch 31. See, for example, FIGS. 2 and 6. In some embodiments, the two grasping pieces 36b of backing material 37 overlap and are connected to the centrally positioned first piece 36a of hook material 37a as shown in FIG. 6. In some embodiments, the two grasping pieces 36b of backing material 37 each independently overlap a distance d₀ (see, FIG. 6) and are connected to end portions 361/362 of the centrally positioned first piece 36a of hook material 37a without being separately bonded to foam arch 31. It should be understood that distance d₀ can be any length of overlap. Typically, distance d₀ is about 2.0 cm (but may be up to about 4.0 cm or more). Any type of connection could be used to connect the two grasping pieces 36b of backing material 37 to the centrally positioned first piece 36a of hook material 37a including, but not limited to, an adhesive bond, a thermal bond (e.g., a heat melt bond), a mechanical bond (e.g., hook/loop material, stitching, a staple, etc.), or any combination thereof. In some embodiments, the two grasping pieces 36b of backing material 37 are connected to the centrally positioned first piece 36a of hook material 37a by stitching.

33. The arm protector kit 10 of any one of embodiments 1 to 32, wherein each tab 40 of said at least three tabs 40 is attachable to, repositionable on, and removable from a major outer surface 24 of said composite pad 20 without causing damage to the major outer surface 24 of said composite pad 20.

34. The arm protector kit 10 of any one of embodiments 1 to 33, wherein each tab 40 of said at least three tabs 40 has a tap thickness S_{T} of less than about 0.3 cm, a tab length S_{L} of from about 15.0 cm to about 28.0 cm, and a tab width S_{W} of from about 3.8 cm to about 10.2 cm.

35. The arm protector kit 10 of any one of embodiments 1 to 34, wherein each tab 40 of said at least three tabs 40 has a tap thickness S_{T} of less than about 0.3 cm, a tab length S_{L} of about 20.0 cm, and a tab width S_{W} of about 5.1 cm.

36. The arm protector kit 10 of any one of embodiments 1 to 35, wherein each tab 40 of said at least three tabs 40 comprises tab hook material 41.

37. The arm protector kit 10 of embodiment 36, wherein said tab hook material 41 comprises nylon hook material. In some embodiments, the nylon hook material comprises a 2″ wide White Hook, 100% nylon hook material.

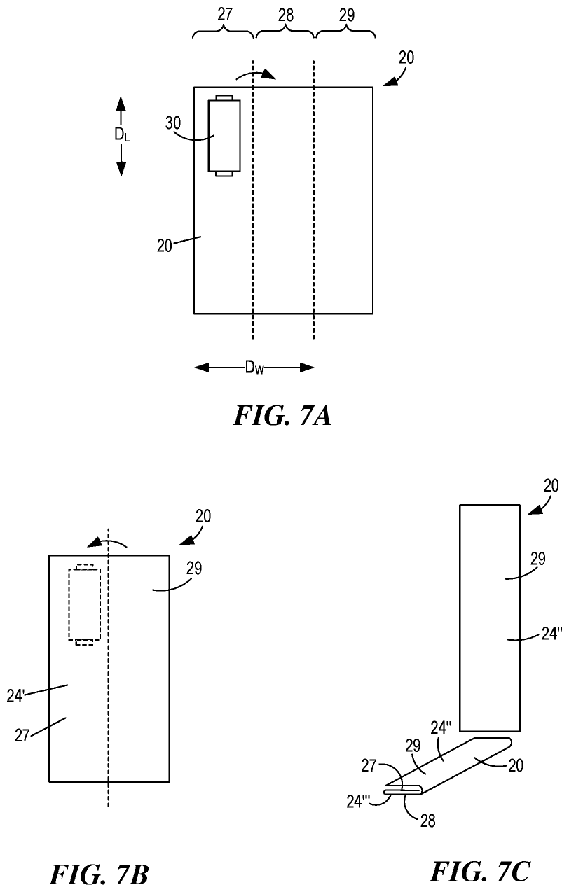

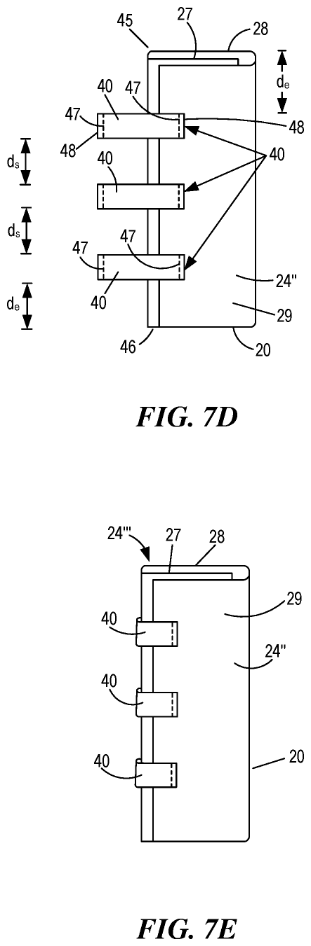

38. The arm protector kit 10 of any one of embodiments 1 to 37, wherein said wrist support component 30 (i) is attachable to, repositionable on, and removable from a first major outer surface 24 of said composite pad 20 and (ii) extending in a length direction D_{L} along an upper, outer third edge 27 of said composite pad 20, and said composite pad 20 is folded along the length direction D_{L} so that the upper, outer third edge 27 of said composite pad 20 is positioned over a central third portion 28 of said composite pad 20. See, for example, FIGS. 7A-7C.

39. The arm protector kit 10 of embodiment 38, wherein an upper, opposite outer third edge portion 29 of said composite pad 20 is folded along the length direction D_{L} so that the upper, opposite outer third edge portion 29 of said composite pad 20 is positioned over the central third portion 28 of said composite pad 20 and a lower surface portion 24′ of the upper, outer third edge portion 27 of said composite pad 20. See again, for example, FIGS. 7A-7C.

40. The arm protector kit 10 of embodiment 39, wherein each tab 40 of said at least three tabs 40 is attachable to, repositionable on, and removable from (i) a lower surface 24″ of the upper, opposite outer third edge portion 29 of said composite pad 20 and (ii) a lower surface 24′″ of the central third portion 28 of said composite pad 20 so as to secure the lower surface 24″ of the upper, opposite outer third edge portion 29 of said composite pad 20 and the lower surface 24′″ of the central third portion 28 of said composite pad 20 to one another. See, for example, FIGS. 7D-7E.

41. The arm protector kit 10 of embodiment 40, wherein the three tabs 40 are independently positioned independent distances dₑ from a distal end 45 and a proximal end 46 of said composite pad 20, and independent separating distances dₛ from one another. See again, for example, FIGS. 7D-7E.

42. The arm protector kit 10 of any one of embodiments 1 to 41, wherein said wrist support component 30 (i) is attachable to, repositionable on, and removable from a central portion 28 of a first major outer surface 24 of said composite pad 20 proximate a distal end 45 of said composite pad 20, and an outer third edge portion 27 of said composite pad 20 is folded along a length direction D_{L} of said composite pad 20 so that the outer third edge portion 27 of said composite pad 20 is positioned over the central portion 28 of said composite pad 20. See, for example, FIGS. 4A-4B.

43. The arm protector kit 10 of embodiment 42, wherein an opposite outer third edge portion 29 of said composite pad 20 is folded along the length direction D_{L} so that the opposite outer third edge portion 29 of said composite pad 20 is positioned over the central portion 28 of said composite pad 20 and a lower surface portion 24′ of the outer third edge portion 27 of said composite pad 20. See, for example, FIG. 4C.

44. The arm protector kit 10 of embodiment 43, wherein two tabs 40 of said at least three tabs 40 are attached to, repositionable on, and removable from (i) a lower surface 24″ of the opposite outer third edge portion 29 of said composite pad 20 and (ii) the lower surface portion 24′ of the outer third edge portion 27 of said composite pad 20 so as to secure the opposite outer third edge portion 29 of said composite pad 20 and the outer third edge portion 27 of said composite pad 20 to one another, said two tabs 40 extending in a width direction D_{W} of said composite pad 20. See again, for example, FIG. 4C.

45. The arm protector kit 10 of embodiment 44, wherein the two tabs 40 are independently positioned independent distances dₑ from a distal end 45 and a proximal end 46 of said composite pad 20, and a separating distance dₛ from one another. See again, for example, FIG. 4C.

46. The arm protector kit 10 of embodiment 44 or 45, wherein one tab 40 of said at least three tabs 40 is attached to, repositionable on, and removable from (i) a lower surface 24′″ of the central portion 28 of said composite pad 20 and (ii) a lower surface 24″ of the opposite outer third edge portion 29 of said composite pad 20 so as to secure the lower surface 24′″ of the central portion 28 of said composite pad 20 and the lower surface 24″ of the opposite outer third edge portion 29 of said composite pad 20 to one another, said one tab 40 extending in a length direction D_{L} of said composite pad 20. See, for example, FIG. 4E.

47. The arm protector kit 10 of any one of embodiments 1 to 46, further comprising one or more numbers and/or letters along one or more major outer surfaces 24 of composite pad 20.

48. The arm protector kit 10 of embodiment 47, wherein said one or more numbers and/or letters comprises: (a) “1R” or “1L” along lower surface 24″ of the upper, opposite outer third edge portion 29 of composite pad 20, indicating that this particular arm protector kit 10 is suitable for use with a right-handed patient 80 or a left-handed patient 80, respectively; (b) “3” or “3↑” along lower surface 24′ of the upper, opposite outer third edge portion 27 of composite pad 20; and (c) “4” or “4↓” along lower surface 24″ of the upper, opposite outer third edge portion 29 of composite pad 20. See, for example, FIG. 9A.

49. The arm protector kit 10 of embodiment 47 or 48, wherein said one or more numbers and/or letters comprises: (d) “2” along major outer surface 24 in an upper, right-hand corner of the upper, opposite outer third edge portion 29 of composite pad 20. See, for example, FIG. 9B.

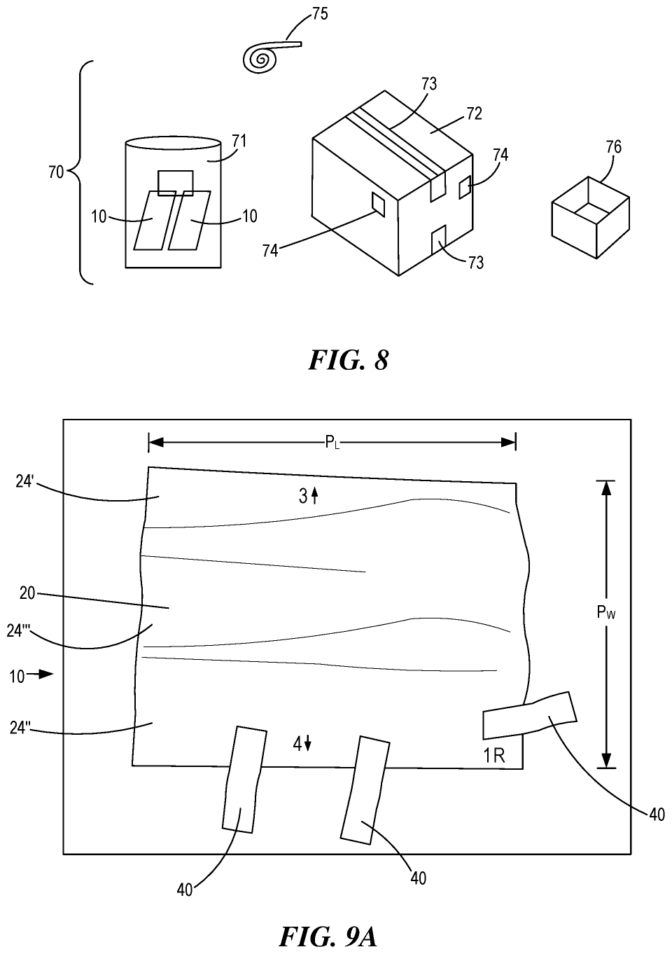

50. The arm protector kit 10 of any one of embodiments 1 to 49, further comprising one or more packaging materials 70 sized to encompass one or more of said arm protector kits 10. Suitable packaging materials 70 include, but are not limited to, a polymeric bag 71, a box 72, tape 73, and labels 74. See, for example, FIG. 8.

51. The arm protector kit 10 of embodiment 50, wherein said one or more packaging materials 70 comprise a polymeric bag 71, a polymeric film 75, tape 73, a shipping container 72, a box 72, a label 74, a crate 76, or any combination thereof.

52. The arm protector kit 10 of any one of embodiments 1 to 49, wherein the arm protector kit 10 is assembled so as to form the protective arm wrapping 50 with the closed end 51 that encapsulates and protects fingers 82 of the patient 80.

Methods of Making Arm Protector Kits:

53. A method of making the arm protector kit 10 of any one of embodiments 1 to 52, said method comprising: combining the composite pad 20, the wrist support component 30, and the at least three tabs 40 with one another.

54. The method of embodiment 53, wherein said combining step comprises attaching the wrist support component 30 and the at least three tabs 40 to the composite pad 20.

55. The method of embodiment 53 or 54, further comprising: a composite pad forming step, said composite pad forming step comprising attaching at least one loop material layer 22 to a major outer surface 23 of the foam layer 21.

56. The method of any one of embodiments 53 to 55, further comprising: a composite pad forming step, said composite pad forming step comprising attaching at least one loop material layer 22 to opposite major outer surfaces 24 of the foam layer 21. In one embodiment, the step of attaching at least one loop material layer 22 to opposite major outer surfaces 24 of the foam layer 21 comprises a flame lamination step.

57. The method of any one of embodiments 53 to 56, further comprising: a wrist support component forming step, said wrist support component forming step comprising forming the foam arch 31.

58. The method of embodiment 57, wherein said wrist support component forming step further comprises attaching a hook backing layer 35 to a lower surface 34 of the foam arch 31.

59. The method of embodiment 58, wherein the hook backing layer 35 comprises up to three separate pieces 36 of backing material 37 with at least one piece 36 of backing material 37 comprising hook material 37a. In some embodiments, the three separate pieces 36 of hook backing material 37 comprise (a) two grasping pieces 36b of backing material 37 that overlap and are connected to a centrally positioned first piece 36a of hook material 37a as shown in FIG. 6. In some embodiments, the two grasping pieces 36b of backing material 37 are connected to end portions 361/362 of the centrally positioned first piece 36a of hook material 37a without being separately bonded to foam arch 31. In some embodiments, the two grasping pieces 36b of backing material 37 are connected to the centrally positioned first piece 36a of hook material 37a by stitching.

60. The method of any one of embodiments 53 to 59, further comprising: a tab forming step, said tab forming step comprising forming tab hook material 41.

61. The method of embodiment 60, wherein said tab forming step further comprises cutting the tab hook material 41 to provide a desired tab length S_{L}.

62. The method of embodiment 61, wherein said tab forming step further comprises stitching a stitch length 47 proximate opposite ends 48 of the tab hook material 41, each stitch length 47 extending in a width direction of the tab hook material 41.

63. The method of any one of embodiments 53 to 62, further comprising: printing one or more numbers and/or letters onto at least one major outer surface 23/24 of the composite pad 20. It should be understood that any method of putting a number and/or letter onto composite pad 20 may be used instead of printing. For example, an embossing step may be used, or an adhering step (e.g., adhering a label onto composite pad 20) may be used.

64. The method of embodiment 63, wherein the one or more numbers and/or letters comprises: (a) “1R” or “1L” along lower surface 24″ of the upper, opposite outer third edge portion 29 of composite pad 20, indicating that this particular arm protector kit 10 is suitable for use with a right-handed patient 80 or a left-handed patient 80, respectively; (b) “3” or “3↑” along lower surface 24′ of the upper, opposite outer third edge portion 27 of composite pad 20; and (c) “4” or “4↓” along lower surface 24″ of the upper, opposite outer third edge portion 29 of composite pad 20. See again, for example, FIG. 9A.

65. The method of embodiment 63 or 64, wherein the one or more numbers and/or letters comprises: (d) “2” along major outer surface 24 in an upper, right-hand corner of the upper, opposite outer third edge portion 29 of composite pad 20. See again, for example, FIG. 9B.

66. The method of any one of embodiments 53 to 65, further comprising: folding the composite pad 20 along the length direction D_{L} so that the outer third edge portion 27 of the composite pad 20 is positioned over a central third portion 28 of the composite pad 20; and folding an opposite outer third edge portion 29 along the length direction D_{L} so that the opposite outer third edge portion 29 is positioned over the central third portion 28 of the composite pad 20 and a lower surface 24′ of the outer third edge portion 27 of the composite pad 20.

67. The method of any one of embodiments 53 to 66, further comprising: packaging the composite pad 20, the wrist support component 30 and the at least three tabs 40 within one or more pieces of packaging material 70.

Methods of Using Arm Protector Kits:

68. A method of using the arm protector kit 10 of any one of embodiments 1 to 52, said method comprising: forming a protective arm wrapping 50 with a closed end 51 that encapsulates and protects fingers 82 of the patient 80.

69. The method of embodiment 68, wherein said forming step takes place during a medical procedure.

70. The method of embodiment 68 or 69, wherein said forming step comprises: positioning the wrist support component 30 so as to attach to a central portion 28 of a first major outer surface 24 of the composite pad 20 proximate a distal end 45 of the composite pad 20. See, for example, FIG. 4A.

71. The method of embodiment 70, wherein said forming step further comprises: positioning the composite pad 20 along and under the patient's arm 81 so that the fingers 82 of the patient 8—extend along an upper surface 38 of the foam arch 31 at a distal end 32 of the foam arch 31. See again, for example, FIG. 4A.

72. The method of embodiment 71, wherein said forming step further comprises: folding an outer edge third portion 27 of the composite pad 20 along a length direction D_{L} of the composite pad 20 so that the outer edge third portion 27 of the composite pad 20 is positioned over the central portion 28 of the composite pad 20. See, for example, FIG. 4B.

73. The method of embodiment 72, wherein said forming step further comprises: folding an opposite outer edge third portion 29 of the composite pad 20 along the length direction D_{L} so that the opposite outer edge third portion 29 of the composite pad 20 is positioned over the central portion 28 of the composite pad 20 and a lower surface portion 24′ of the outer edge third portion 27 of the composite pad 20. See, for example, FIG. 4C.

74. The method of embodiment 73, wherein said forming step further comprises: attaching two tabs 40 of the at least three tabs 40 to the composite pad 20 so as to secure (i) the opposite outer edge third portion 29 of the composite pad 20 and (ii) the outer edge third portion 27 of the composite pad 20 to one another, the two tabs 40 extending in a width direction D_{W} of the composite pad 20. See again, for example, FIG. 4C.

75. The method of embodiment 74, wherein the two tabs 40 are independently positioned independent distances dₑ from a distal end 45 and a proximal end 46 of the composite pad 20, and a separating distance dₛ from one another. See again, for example, FIG. 4C.

76. The method of embodiment 74 or 75, wherein said forming step further comprises: attaching one tab 40 of the at least three tabs 40 to the composite pad 20 so as to secure (i) a lower surface 24′″ of the central portion 28 of the composite pad 20 and (ii) a lower surface 24″ of the opposite outer third edge portion 29 of the composite pad 20 to one another, the one tab 40 extending in a length direction D_{L} of the composite pad 20 and forming the closed end 51 of the protective arm wrapping 50.

77. The method of any one of embodiments 68 to 76, further comprising: cutting the composite pad 20 to shorten the pad length P_{L}.

78. The method of any one of embodiments 68 to 77, further comprising: repositioning (i) the wrist support component 30, (ii) at least one tab 40 of the at least three tabs 40, or (iii) both (i) the wrist support component 30 and (ii) at least one tab 40 of the at least three tabs 40 along a major outer surface 24 of the composite pad 20.

79. The method of any one of embodiments 68 to 78, further comprising: removing (i) the wrist support component 30, (ii) at least one tab 40 of the at least three tabs 40, or (iii) both (i) the wrist support component 30 and (ii) at least one tab 40 of the at least three tabs 40 from a major outer surface 24 of the composite pad 20.

80. The method of any one of embodiments 68 to 79, further comprising: attaching (i) the wrist support component 30, (ii) at least one tab 40 of the at least three tabs 40, or (iii) both (i) the wrist support component 30 and (ii) at least one tab 40 of the at least three tabs 40 to a major outer surface 24 of the composite pad 20.

81. The method of any one of embodiments 68 to 80, further comprising: removing (i) the composite pad 20, (ii) the wrist support component 30, and (iii) the at least three tabs 40 from one or more packaging materials 70.

82. The method of any one of embodiments 68 to 81, further comprising: discarding (i) the composite pad 20, (ii) the wrist support component 30, and (iii) the at least three tabs 40 after a single use.

The present invention is described above and further illustrated below by way of examples, which are not to be construed in any way as imposing limitations upon the scope of the invention. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### Example 1

### Preparation of Arm Protector Kits

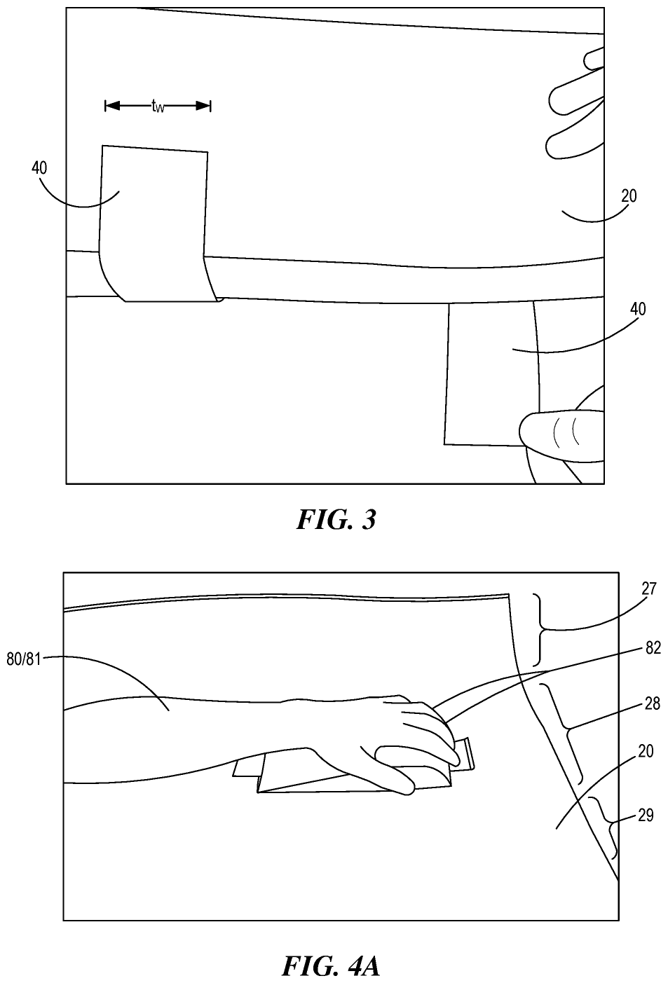

Exemplary arm protector kits 10 as shown in FIGS. 1-10E were prepared using conventional steps (e.g., one or more thermoforming steps, and one or more connection/assembly steps, one or more laminating steps, one or more cutting steps, etc.).

### Example 2

### Method of Using Arm Protector Kits

The exemplary arm protector kits 10 formed in Example 1 were used to form an arm protective wrapping 50 over a patient's arm 80 using the procedure shown in FIGS. 4A-4E, and FIG. 10A-10E.

It should be understood that although the above-described arm protector kits and methods are described as “comprising” one or more components or steps, the above-described arm protector kits and methods may “comprise,” “consists of,” or “consist essentially of” any of the above-described components, features or steps of the arm protector kits and methods. Consequently, where the present invention, or a portion thereof, has been described with an open-ended term such as “comprising,” it should be readily understood that (unless otherwise stated) the description of the present invention, or the portion thereof, should also be interpreted to describe the present invention, or a portion thereof, using the terms “consisting essentially of” or “consisting of” or variations thereof as discussed below.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, an arm protector kit and/or method that “comprises” a list of elements (e.g., components, features or steps) is not necessarily limited to only those elements (or components or steps), but may include other elements (or components or steps) not expressly listed or inherent to the arm protector kit and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a arm protector kit and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Further, it should be understood that the herein-described arm protector kits and/or methods may comprise, consist essentially of, or consist of any of the herein-described components and features, as shown in the figures with or without any feature(s) not shown in the figures. In other words, in some embodiments, the arm protector kits and/or methods of the present invention do not have any additional features other than those shown in the figures, and such additional features, not shown in the figures, are specifically excluded from the arm protector kits and/or methods. In other embodiments, the arm protector kits and/or methods of the present invention do have one or more additional features that are not shown in the figures.

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. An arm protector kit comprising:
a composite pad sized to extend around and along an arm of a patient, said composite pad having a pad thickness, a pad length and a pad width, and comprising a foam layer and at least one loop material layer along a major outer surface of the foam layer;
a wrist support component that is attachable to, repositionable on, and removable from a major outer surface of said composite pad, said wrist support component comprising a foam arch; and
at least three tabs, each tab (i) being attachable to, repositionable on, and removable from a major outer surface of said composite pad, and (ii) comprising a strip of hook material having a strip length and a strip width,
wherein the arm protector kit forms a protective arm wrapping with a closed end that encapsulates and protects fingers of the patient when assembled on the arm of the patient.

2. The arm protector kit of claim 1, wherein said foam layer comprises a single foam layer having (i) a layer density of from about 0.5 to about 5.0 lb density, (ii) an Impression Load Deflection (ILD) value of from about 36 to about 50, and (iii) a layer thickness t_{FOL} of from about 0.5 centimeter (cm) to about 2.0 cm.

3. The arm protector kit of claim 2, wherein the foam layer comprises a polyurethane non-viscoelastic foam, a polyester non-viscoelastic foam, a polyester polyurethane non-viscoelastic foam, or a combination thereof.

4. The arm protector kit of claim 1, further comprising an adhesive layer between the foam layer and each of said at least one loop material layer.

5. The arm protector kit of claim 1, wherein each of said at least one loop material layer comprises a warp unbroken loop (UBL) knit fabric.

6. The arm protector kit of claim 1, wherein said composite pad has a pad thickness of from about 0.5 cm to about 2.0 cm, a pad length of from about 50.0 cm to about 90.0 cm, and a pad width of from about 40.0 cm to about 70.0 cm.

7. The arm protector kit of claim 1, wherein said wrist support component is attachable to, repositionable on, and removable from a major outer surface of said composite pad without causing damage to the major outer surface of said composite pad, and said foam arch has an arch maximum thickness of from about 2.5 cm to about 6.0 cm, an arch length of from about 15.0 cm to about 28.0 cm, and an arch width of from about 6.0 cm to about 15.0 cm.

8. The arm protector kit of claim 7, wherein said foam arch has an arch maximum thickness of about 4.0 cm, an arch length of about 21.0 cm, and an arch width of about 10.0 cm, and said arch maximum thickness is closer to a distal end of said foam arch than a proximal end of said foam arch.

9. The arm protector kit of claim 7, wherein said foam arch has an arch upper surface profile that gradually increases in height from a proximal end of said foam arch to a point along the arch length of said foam arch that contains the arch maximum thickness of said foam arch, and then curves downward towards a distal end of said foam arch.

10. The arm protector kit of claim 1, wherein said foam arch (a) has an arch upper surface profile that forms (i) a first angle A₁ with a lower surface of said foam arch at a proximal end of said foam arch, and (ii) a second angle A₂ with the lower surface of said foam arch at a distal end of said foam arch, and wherein the first angle A₁ is less than about 15° and the second angle A₂ is from about 60° to about 90°, and (b) comprises a polyurethane non-viscoelastic foam, a polyester non-viscoelastic foam, a polyester polyurethane non-viscoelastic foam, or a combination thereof.

11. The arm protector kit of claim 1, wherein said wrist support component further comprises a hook backing layer, said hook backing layer (i) extending along a lower surface of said foam arch, (ii) comprising one or more separate pieces of backing material, wherein at least one piece of backing material comprises a hook material.

12. The arm protector kit of claim 11, wherein said hook backing layer comprises (1) a centrally positioned first piece of backing material comprising the hook material with (i) a backing material length L_{HBM} of about 17.0 cm and (ii) a backing material width W_{HBM} of about 5.0 cm, and (2) two grasping pieces of backing material, each independently comprising a fiber webbing having (i) a backing material length L_{HBM} of about 7.0 cm and (ii) a backing material width W_{HBM} of about 5.0 cm, wherein each of the two grasping pieces of backing material extend beyond a lower surface of the foam arch at opposite distal and proximal ends of said foam arch.

13. The arm protector kit of claim 1, wherein each tab of said at least three tabs (i) is attachable to, repositionable on, and removable from a major outer surface of said composite pad without causing damage to the major outer surface of said composite pad, (ii) has a tap thickness of less than about 0.3 cm, a tab length of from about 15.0 cm to about 28.0 cm, and a tab width of from about 3.8 cm to about 10.2 cm.

14. The arm protector kit of claim 13, wherein said tab hook material comprises a nylon hook material.

15. The arm protector kit of claim 1, wherein said wrist support component (i) is attachable to, repositionable on, and removable from a central portion of a first major outer surface of said composite pad proximate a distal end of said composite pad, and an outer third edge portion of said composite pad is folded along a length direction D_{L} of said composite pad so that the outer third edge portion of said composite pad is positioned over the central portion of said composite pad.

16. The arm protector kit of claim 15, wherein an opposite outer third edge portion of said composite pad is folded along the length direction D_{L} so that the opposite outer third edge portion of said composite pad is positioned over the central portion of said composite pad and a lower surface portion of the outer third edge portion of said composite pad.

17. The arm protector kit of claim 16, wherein two tabs of said at least three tabs are attached to, repositionable on, and removable from (i) a lower surface of the opposite outer third edge portion of said composite pad and (ii) the lower surface portion of the outer third edge portion of said composite pad so as to secure the opposite outer third edge portion of said composite pad and the outer third edge portion of said composite pad to one another, said two tabs extending in a width direction D_{W} of said composite pad.

18. The arm protector kit of claim 17, wherein one tab of said at least three tabs is attached to, repositionable on, and removable from (i) a lower surface of the central portion of said composite pad and (ii) a lower surface of the opposite outer third edge portion of said composite pad so as to secure lower surface of the central portion of said composite pad and lower surface of the opposite outer third edge portion of said composite pad to one another, said one tab extending in a length direction D_{L} of said composite pad.

19. A method of using the arm protector kit of claim 1, said method comprising:
forming a protective arm wrapping with a closed end that encapsulates and protects fingers of the patient, wherein said forming step comprises:
positioning the wrist support component so as to attach to a central portion of a first major outer surface of the composite pad proximate a distal end of the composite pad;
positioning the composite pad along and under the patient's arm so that the fingers of the patient extend along an upper surface of the foam arch at a distal end of the foam arch;
folding an outer third edge portion of the composite pad along a length direction D_{L} of the composite pad so that the outer third edge portion of the composite pad is positioned over the central portion of the composite pad;
folding an opposite outer third edge portion of the composite pad along the length direction D_{L} so that the opposite outer third edge portion of the composite pad is positioned over the central portion of the composite pad and a lower surface portion of the outer third edge portion of the composite pad;
attaching two tabs of the at least three tabs to the composite pad so as to secure (i) the opposite outer third edge portion of the composite pad and (ii) the outer third edge portion of the composite pad to one another, the two tabs extending in a width direction D_{W} of the composite pad, wherein the two tabs are independently positioned independent distances from a distal end and a proximal end of the composite pad, and a separating distance from one another; and
attaching one tab of the at least three tabs to the composite pad so as to secure (i) a lower surface of the central portion of the composite pad and (ii) a lower surface of the opposite outer third edge portion of the composite pad to one another, the one tab extending in a length direction D_{L} of the composite pad and forming the closed end of the protective arm wrapping.

20. The method of claim 19, further comprising:
discarding (i) the composite pad, (ii) the wrist support component, and (iii) the at least three tabs after a single use.
